(19) United States
(12) Patent Application Publication
(10) Pub. No.: US 2026/0226508 A1
(43) Pub. Date: Aug. 6, 2026

(54) GENETIC CONTROL OF MRI SIGNAL

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Harikrishna RALLAPALLI, Bethesda, MD (US); Alan Paul KORETSKY, Bethesda, MD (US); Raymond Lee FIELDS, Bethesda, MD (US)

(21) Appl. No.: 19/147,173

(22) PCT Filed: Jan. 12, 2024
(86) PCT No.: PCT/US2024/011377

§ 371 Date: Jul. 10, 2025

Related U.S. Application Data
(60) Provisional application No. 63/479,835, filed on Jan. 13, 2023.

(51) Int. Cl. C12N 15/864; A61K 49/18; C07K 14/47; C12N 15/861; C12N 15/867; C12N 15/88

(52) U.S. Cl. CPC C12N 15/8645; A61K 49/1824; C07K 14/47; C12N 15/861; C12N 15/867; C12N 15/88; C12N 2710/10043; C12N 2740/15043; C12N 2750/14143; C12N 2830/34

(57) ABSTRACT

MRI visible reporter systems comprising ZIP14 and/or ZIP8 and methods of use.

### CROSS-REFERENCE TO RELATED APPLICATIONS

This international patent application claims priority to U.S. Provisional Patent Application No. 63/479,835, filed Jan. 13, 2023, the contents of which are hereby incorporated by reference in its entirety.

### REFERENCE TO SEQUENCE LISTING SUBMITTED ELECTRONICALLY

Pursuant to the legal framework for Patent Electronic System and 37 CFR §§ 1.821-825 (see MPEP § 2442.03(a)), a Sequence Listing in the form of an XML-compliant file (entitled “3000097-001977-Sequence-Listing” created on 12 Jan. 2024 and 11,848 bytes in size) is submitted concurrently with the instant application, and the entire contents of the Sequence Listing are incorporated herein by reference.

### BACKGROUND

### 1. Field

The present disclosure relates to magnetic resonance imaging (MRI) signal changes by controlled overexpression of Zip14. The Zip14 constructs described herein can be used for imaging gene therapy and regenerative medicine methods as well as other areas where an in vivo gene expression strategy is required.

### 2. Description of Related Art

Current Magnetic Resonance Imaging (MRI) visible reporter systems all require modification of tissue in vitro then implantation, addition of exogenous contrast agents in addition to the reporter system, and/or the use of non-standard imaging sequences. There exists a need in the art for targeting and non-invasive tracking of gene therapies and real-time in vivo modeling.

### BRIEF SUMMARY OF THE INVENTION

In an embodiment, an isolated polynucleotide comprising a tissue-specific promoter operably coupled to a Zip14 encoding sequence having at least about 90% homology to the polynucleotide sequence of SEQ ID NO:1. The promoter and/or the Zip14 sequence can be recombinant. The Zip14 sequence can be codon-optimized, optionally for humans. The polynucleotide can further comprise an exogenous gene. The polynucleotide can further comprise a second reporter fused to the Zip14. The reporter gene can be a fluorescent protein for fluorescence detection in histology or a short epitope tag.

In an embodiment, for imaging in the brain the tissue-specific promoter can be a neuronal specific promoter. The neuronal specific promoter can be a Synapsin, Calbindin, Pax6, Sox2, neurofilament heavy chain (NEFH), Arc, or cFos promoter. The tissue-specific promoter can be an astrocyte specific promoter. The astrocyte specific promoter can be a GFAP or S100b promoter. The tissue-specific promoter can be a microglia specific promoter. The microglia specific promoter can be Iba1 promoter. The tissue-specific promoter can be a blood vessel specific promoter. The blood vessel specific promoter can be Rosa or CAG promoter. The promoters of this embodiment can also be used in the other embodiments. Similarly other organ specific cell type promotors can be used for example heart or skeletal muscle. The tissue specific promoter can be a cardiomyocyte specific promoter. Cardiomyocyte specific promoters can be alpha-myosin heavy chain or beta-myosin heavy chain.

In an embodiment, the Zip14 sequence comprises a polynucleotide with at least about 90% sequence homology to the polynucleotide sequence of SEQ ID NO: 1. The Zip14 sequence comprises a polynucleotide with at least about 91%, 92%, 93%, 94%, 95%, 96%, 97%, 98%, 99%, or 100% sequence identity to the polynucleotide sequence of SEQ ID NO: 1. The Zip14 sequences of this embodiment can also be used in the other embodiments.

In an embodiment, an isolated vector can comprise the Zip14 construct polynucleotide described herein. The vector can be Bacterial Artificial Chromosome (BAC), Yeast Artificial Chromosome (YAC), or a plasmid. The vector can be plasmid. The vector can be formulated with lipid nanoparticles.

In an embodiment, the Zip8 sequence comprises a polynucleotide with at least about 90% sequence homology to the polynucleotide sequence of SEQ ID NO: 2, 3, or 5. The Zip8 sequence comprises a polynucleotide with at least about 91%, 92%, 93%, 94%, 95%, 96%, 97%, 98%, 99%, or 100% sequence identity to the polynucleotide sequence of SEQ ID NO: 2, 3, or 5. The Zip8 sequences of this embodiment can also be used in the other embodiments.

In an embodiment, an isolated vector can comprise the Zip8 construct polynucleotide described herein. The vector can be Bacterial Artificial Chromosome (BAC), Yeast Artificial Chromosome (YAC), or a plasmid. The vector can be plasmid. The vector can be formulated with lipid nanoparticles.

The vector can be a viral vector. The vector can be a retroviral vector, an adenoviral vector (AdV), an adeno-associated viral vector (AAV), or a lentiviral vector. The vector can be a AAV1, AAV2, AAV3, AAV4, AAV5, AAV6, AAV7, AAV8, or a AAV9 serotype adenoviral-associated vector. The vector can be a AAV9 vector. The vector can be a retroAAV vector. The vector can be PHP.eB, PHP.S, or PHP.V1 serotype AAV vector. The vector can be a PHPeB AAV vector. The vectors of this embodiment can also be used in the other embodiments.

In an embodiment, a method of monitoring gene expression in a subject can comprise administering a composition comprising an effective amount the polynucleotide described herein or the vector described herein to a subject in need thereof and evaluating the subject using an imaging method. The method of this embodiment can also be used with the other embodiments.

In an embodiment, a method of visualizing a tissue in a subject can comprise administering a composition comprising an effective amount the polynucleotide described herein or the vector described herein to a subject in need thereof and evaluating the subject using an imaging method. The method can further comprise determining cell number, cell density, cell health, cell death, or a combination thereof. The method of this embodiment can also be used with the other embodiments.

The method can further comprise tissue visualization, tissue expression, tissue mapping, monitoring trauma, and cell death, and/or tracking gene therapy delivery and expression via the imaging method. The imaging method can be selected from Magnetic Resonance Imaging (MRI), computerized tomography (CT) scans, Positron Emission Tomography (PET), or a combination thereof. The imaging method can be Magnetic Resonance Imaging (MRI).

In an embodiment, the administration can be subcutaneous, intramuscular, intravenous, intraperitoneal, intrapleural, intravesicular, intrathecal, intracerebroventricular, intranasal injection, or a combination thereof. The administration described by this embodiment can also be used in the other embodiments.

In an embodiment, the administration can be intravenous. The polynucleotide described herein or the vector described herein crosses the blood-brain barrier (BBB) after administration in the case of the brain.

In an embodiment, the composition comprises between about 1×10¹⁰ vg/mL (viral genome per mL) and 1×10¹⁴ vg/mL of the vector. The composition can comprise about 1×10¹⁰ vg/mL, 1×10¹¹ vg/mL, 1×10¹² vg/mL, 1×10¹³ vg/mL, or 1×10¹⁴ vg/mL of the vector. The composition can comprise about 1×10¹² vg/mL of the vector. The vg/mL of this embodiment can also be used in the other embodiments.

In an embodiment, the tissue is the central nervous system and the viral vector serotype is AAV1, AAV2, AAV4, AAV5, AAV8, or AAV9.

In an embodiment, the tissue is the cardiac tissue and the viral vector serotype is AAV1, AAV8, or AAV9.

In an embodiment, the tissue is the kidney and the viral vector serotype is AAV2.

In an embodiment, the tissue is the liver and the viral vector serotype is AAV7, AAV8, or AAV9.

In an embodiment, the tissue are the lungs and the viral vector serotype is AAV4, AAV5, AAV6, and AAV9.

In an embodiment, the tissue is the pancreas and the viral vector serotype is AAV8.

In an embodiment, the tissue photoreceptor cells and the viral vector serotype is AAV2, AAV5, and AAV8.

In an embodiment, the tissue is the RPE (Retinal Pigment Epithelium) and the viral vector serotype is AAV1, AAV2, AAV4, AAV5, and AAV8.

In an embodiment, the tissue is the skeletal muscle and the viral serotype is AAV1, AAV6, AAV7, AAV8, and AAV9.

The tissues and viral serotypes of the above embodiments can also be used in the other embodiments.

In an embodiment, the method can further comprise using machine learning to map the area detectable by Zip14 expression. The machine learning of this embodiment can also be used in the other embodiments. Machine learning can be used to classify the data to more readily identify the signal produced by Zip8 and/or Zip14. The machine learning classifiers can be AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, deep neural networks, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof.

### BRIEF DESCRIPTION OF THE DRAWINGS

FIG. 1 depicts Zip14 overexpression produces long-lasting signal enhancement on MRI. (A) Coronal MRI image of a mouse brain immediately after injection with AAV-Zip14-EGFP into the right S1 barrel cortex (white circle, S1BC). (B) Coronal MRI image of the same animal 2 weeks after injection shows focal hyperintensity in the S1BC (white circle). (C) Coronal MRI image of the same animal 4 weeks after injection shows that the focal, high-magnitude signal increase due to Zip14 overexpression is persistent. (D) Immunofluorescence histology shows focal overexpression of Zip14, validating the source of enhancement apparent in the cortex (white circle).

FIG. 2 depicts AAV-Zip14-EGFP provides signal enhancement at the injection site and in projecting areas. (A) Schematic diagram of anterograde connection from S1BC to ventral posteromedial nucleus of the thalamus (VPM); (B) Coronal MRI image of an animal injected with AAV-Zip14-EGFP into the S1BC shows bright signal at the injection site (white circle) and focal bright signal in the VPM (white square); (C) Immunofluorescence imaging for Zip14-EGFP corroborates that the bright signal in the S1BC (white circle) and VPM (white square) are due to Zip14 overexpression in those areas.

FIG. 3 depicts retroAAV-Zip14 potentiates tracing parts of the cortico-basal ganglia-thalamic network. (A) depicts schematic of the cortico-basal ganglia-thalamic network; (B) At 0 weeks post injection into the CP, no signal enhancement was apparent at the injection site (white bar, top row) or posterior sections (bottom row); (C) At 2 weeks post injection, there was large magnitude signal enhancement apparent in the CP and posterior nuclei. (injection site—top and posterior section—bottom); (D) Subtraction of the 2 week post injection image from the 0 week post injection image highlights areas of enhancement due to Zip14 overexpression (injection site—top and posterior section—bottom).

FIG. 4 depicts Zip14 overexpression highlights the growth of brain organoids. (A) Axial MRI image of a rat with a brain organoid growing in the motor cortex, 2 weeks after PHPeB-Zip14-FLAG injection into the organoid (white circle); (B) Coronal MRI image at the level of the organoid (white circle) of the same animal shows focal bright signal; (C) Immunofluorescence histology for Zip14-FLAG and EGFP (organoid) confirms that the bright signal seen in the organoid in MRI is due to Zip14 overexpression.

### DETAILED DESCRIPTION

Before the subject disclosure is further described, it is to be understood that the disclosure is not limited to the particular embodiments of the disclosure described below, as variations of the particular embodiments can be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“Effective amount,” as used herein, refers broadly to the amount of a compound, antibody, antigen, or cells that, when administered to a patient for treating a disease, is sufficient to effect such treatment for the disease. The effective amount can be an amount effective for prophylaxis, and/or an amount effective for prevention. The effective amount can be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

“Treatment,” as used herein, refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

The term “variant” as used herein refers broadly to a polypeptide that possesses a similar or identical function as a Zip14 polypeptide but does not necessarily comprise a similar or identical amino acid sequence of a Zip14 polypeptide. A variant having a similar amino acid identity refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of a Zip14 polypeptide; (b) a polypeptide encoded by a nucleotide sequence, the complementary sequence of which hybridizes under stringent conditions to a nucleotide sequence encoding a Zip14 polypeptide, of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, identical to the nucleotide sequence encoding a Zip14. A polypeptide with similar structure to a Zip14 polypeptide described herein refers to a polypeptide that has a similar secondary, tertiary or quaternary structure of a Zip14 polypeptide. The structure of a polypeptide can be determined by methods known to those skilled in the art, including but not limited to, X-ray crystallography, nuclear magnetic resonance, and crystallographic electron microscopy. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (e.g., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268 (1990), modified as in Karlin and Altschul Proc. Natl. Acad. Sci. USA 90:5873-5877 (1993). The BLASTn and BLASTx programs of Altschul, et al. J. Mal. Biol. 215:403-410 (1990) have incorporated such an algorithm. BLAST nucleotide searches can be performed with the BLASTn program, score=100, wordlength=12 to obtain nucleotide sequences homologous to a nucleic acid molecule described herein. BLAST protein searches can be performed with the BLASTx program, score=50, wordlength=3 to obtain amino acid sequences homologous to a protein molecule described herein. To obtain gapped alignments for comparison purposes, Gapped BLAST can be utilized as described in Altschul et al. Nucleic Acids Res. 25:3389-3402 (1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used. (See ncbi.nlm.nih.gov).

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci., 10:3-5(1994); and FASTA described in Pearson and Lipman Proc. Natl. Acad. Sci. 85:2444-8(1988). Within FASTA, ktup is a control option that sets the sensitivity and speed of the search.

“Conservative” amino acid substitutions are those substitutions that do not substantially affect or decrease the activity of Zip14 and can include at most about 1, at most about 2, at most about 5, at most about 10, or at most about 15 conservative substitutions and still retain the activity of the unmodified Zip14. The term “conservative variant” also includes the use of a substituted amino acid in place of an unsubstituted parent amino acid, provided that polypeptide retains the activity of the unmodified Zip14. Non-conservative substitutions are those that reduce the activity Zip14 as compared to wild-type Zip14.

Conservative amino acid substitution tables providing functionally similar amino acids are well known to one of ordinary skill in the art. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

### Zip8 and Zip14 Constructs

The inventors developed a method to produce magnetic resonance imaging (MRI) signal changes by controlled overexpression of Zip14.

Zip14 (SLC39A14) and Zip8 (SLC39A8) are two members of the SLC39A family of proteins involved in the transport of many metal ions including iron, zinc, and manganese across cellular membranes. Zip14 and Zip8 are closely related and structurally similar: murine Zip14 and Zip8 are 489 and 462 amino acids in length, respectively, and ~50% identical in amino acid sequence. Both are functionally implicated in manganese uptake in the brain and liver. Zip8 works analogously to Zip14 in the methods described herein.

### Zip14

A DNA plasmid constructs consisting of a promoter of interest (e.g., hSyn for neuron-specific signal) operably coupled to Slc39a14 (the gene coding for Zip14), and in line for expression of a molecular tag (e.g., GFP or FLAG) (under control of the same promoter) for histological validation were packaged into adeno-associated viruses (Zip14-AAVs).

These Zip14-AAVs were delivered focally and systemically into small animal models. MRI was performed immediately after and at various timepoints after injection. The methods described herein produces robust, long-lasting, specific signal changes in vivo. The neuron-specific Zip14 constructs described herein can be used in methods including but not limited to long-range tracing, gene therapy efficacy studies, and regenerative medicine in the context of brain injury. Zip14 constructs with other tissue specific promoters can be used in other cell types and report expression of genes of interest.

The Zip14 sequence can be a polynucleotide sequence with at least about 90% homology to the polynucleotide sequence of SEQ ID NO:1:

ATGAAGCTGCTGCTGCTGCACCCGGCCTTCCAGAGCTGCCTCCTGCTGACCCTGC
TTGGCTTATGGAGAACCACCCCTGAGGCTCACGCTTCATCCCTGGGTGCACCAGC
TATCAGCGCTGCCTCCTTCCTGCAGGATCTAATACATCGGTATGGCGAGGGTGAC
AGCCTCACTCTGCAGCAGCTGAAGGCCCTACTCAACCACCTGGATGTGGGAGTG
GGCCGGGGTAATGTCACCCAGCACGTGCAAGGACACAGGAACCTCTCCACGTGC
TTTAGTTCTGGAGACCTCTTCACTGCCCACAATTTCAGCGAGCAGTCGCGGATTG
GGAGCAGCGAGCTCCAGGAGTTCTGCCCCACCATCCTCCAGCAGCTGGATTCCCG
GGCCTGCACCTCGGAGAACCAGGAAAACGAGGAGAATGAGCAGACGGAGGAGG
GGCGGCCAAGCGCTGTTGAAGTGTGGGGATACGGTCTCCTCTGTGTGACCGTCAT
CTCCCTCTGCTCCCTCCTGGGGGCCAGCGTGGTGCCCTTCATGAAGAAGACCTTT
TACAAGAGGCTGCTGCTCTACTTCATAGCTCTGGCGATTGGAACCCTCTACTCCA
ACGCCCTCTTCCAGCTCATCCCGGAGGCATTTGGTTTCAACCCTCTGGAAGATTA
TTATGTCTCCAAGTCTGCAGTGGTGTTTGGGGGCTTTTATCTTTTCTTTTTCACAG
AGAAGATCTTGAAGATTCTTCTTAAGCAGAAAAATGAGCATCATCATGGACACA
GCCATTATGCCTCTGAGTCGCTTCCCTCCAAGAAGGACCAGGAGGAGGGGGTGA
TGGAGAAGCTGCAGAACGGGGACCTGGACCACATGATTCCTCAGCACTGCAGCA
GTGAGCTGGACGGCAAGGCGCCCATGGTGGACGAGAAGGTCATTGTGGGCTCGC
TCTCTGTGCAGGACCTGCAGGCTTCCCAGAGTGCTTGCTACTGGCTGAAAGGTGT
CCGCTACTCTGATATCGGCACTCTGGCCTGGATGATCACTCTGAGCGACGGCCTC
CATAATTTCATCGATGGCCTGGCCATCGGTGCTTCCTTCACTGTGTCAGTTTTCCA
AGGCATCAGCACCTCGGTGGCCATCCTCTGTGAGGAGTTCCCACATGAGCTAGGA
GACTTTGTCATCCTGCTCAACGCTGGGATGAGCATCCAACAAGCTCTCTTCTTCA
ACTTCCTTTCTGCCTGCTGCTGCTACCTGGGTCTGGCCTTTGGCATCCTGGCCGGC
AGCCACTTCTCTGCCAACTGGATTTTTGCGCTAGCTGGAGGAATGTTCTTGTATAT
TTCTCTGGCTGATATGTTCCCTGAGATGAATGAGGTCTGTCAAGAGGATGAAAGG
AAGGGCAGCATCTTGATTCCATTTATCATCCAGAACCTGGGCCTCCTGACTGGAT
TCACCATCATGGTGGTCCTCACCATGTATTCAGGACAGATCCAGATTGGG

Zip14 sequences that can be used in the constructs and methods described herein can be found at: uniprot.org/uniprotkb/Q75N73/entry#phenotypes_variants.

Additional embodiments described herein are directed to polynucleotides comprising, or alternatively consisting of, a polynucleotide having a polynucleotide sequence of about 90% to 99% sequence identity to a Zip14 polynucleotide having the polynucleotide sequence of SEQ ID NO: 1. The Zip14 polynucleotide can have at least about 91%, 92%, 93%, 94%, 95%, 96%, 97%, 98%, 99%, or 100% sequence identity to the polynucleotide sequence of SEQ ID NO: 1.

### Zip8

A DNA plasmid constructs consisting of a promoter of interest (e.g., hSyn for neuron-specific signal) operably coupled to SLC39A8 (the gene coding for Zip8), and in line for expression of a molecular tag (e.g., GFP or FLAG) (under control of the same promoter) for histological validation can be packaged into adeno-associated viruses (Zip8-AAVs).

These Zip8-AAVs can be delivered focally and systemically into small animal models. MRI can be performed immediately after and at various timepoints after injection. The methods described herein produces robust, long-lasting, specific signal changes in vivo. The neuron-specific Zip8 constructs described herein can be used in methods including but not limited to long-range tracing, gene therapy efficacy studies, and regenerative medicine in the context of brain injury. Zip8 constructs with other tissue specific promoters can be used in other cell types and report expression of genes of interest.

The Zip8 sequence can be a polynucleotide sequence with at least about 90% homology to the polynucleotide sequence of SEQ ID NO:5:

ATGGCCCCTGGCCGGGCCGTGGCCGGCCTGCTGCTGCTGGCAGCAGCAGGACTG
GGAGGAGTGGCCGAGGGCCCAGGCCTGGCCTTTTCCGAGGACGTGCTGAGCGTG
TTCGGAGCAAACCTGTCCCTGTCTGCCGCACAGCTGCAGCACCTGCTGGAGCAGA
TGGGAGCAGCATCCAGAGTGGGAGTGCCAGAGCCTGGCCAGCTGCACTTCAACC
AGTGTCTGACCGCCGAGGAGATCTTTAGCCTGCACGGCTTCTCCAATGCCACCCA
GATCACAAGCTCCAAGTTTTCTGTGATCTGCCCCGCCGTGCTGCAGCAGCTGAAC
TTCCACCCGTGCGAGGATAGGCCGAAACATAAGACAAGACCTAGCCACTCCGAA
GTGTGGGGCTACGGCTTTCTGTCTGTGACCATCATCAATCTGGCCAGCCTGCTGG
GCCTGATCCTGACACCACTGATCAAGAAGTCCTATTTCCCCAAGATCCTGACCTT
CTTTGTGGGCCTGGCCATCGGCACACTGTTTTCTAACGCCATCTTCCAGCTGATCC
CCGAGGCCTTTGGCTTCGACCCTAAGGTGGATAGCTACGTGGAGAAGGCCGTGG
CCGTGTTTGGCGGCTTCTATCTGCTGTTCTTTTTCGAGAGGATGCTGAAGATGCTG
CTGAAGACCTACGGCCAGAATGGCCACACACACTTTGGCAACGACAATTTCGGC
CCCCAGGAGAAGACCCACCAGCCAAAGGCCCTGCCAGCCATCAACGGAGTGACC
TGCTATGCCAATCCTGCCGTGACAGAGGCCAACGGCCACATCCACTTTGACAACG
TGAGCGTGGTGTCTCTGCAGGATGGCAAGAAGGAGCCTTCTAGCTGCACCTGTCT
GAAGGGCCCAAAGCTGTCCGAGATCGGCACCATCGCCTGGATGATCACACTGTG
CGACGCCCTGCACAACTTCATCGATGGCCTGGCCATCGGCGCCTCTTGTACCCTG
AGCCTGCTGCAGGGCCTGTCTACAAGCATCGCCATCCTGTGCGAGGAGTTTCCCC
ACGAGCTGGGCGACTTCGTGATCCTGCTGAACGCCGGCATGTCCACCAGACAGG
CCCTGCTGTTTAATTTCCTGAGCGCCTGCTCCTGTTACGTGGGACTGGCCTTCGGC
ATCCTGGTGGGCAACAATTTTGCCCCAAATATCATCTTCGCCCTGGCCGGCGGCA
TGTTTCTGTATATCAGCCTGGCCGACATGTTCCCCGAGATGAACGATATGCTGAG
GGAGAAGGTGACCGGCCGCAAGACCGACTTCACCTTCTTCATGATCCAGAATGC
CGGCATGCTGACCGGCTTCACAGCCATCCTGCTGATCACACTGTACGCCGGCGAG
ATCGAGCTGGAG

Additional embodiments described herein are directed to polynucleotides comprising, or alternatively consisting of, a polynucleotide having a polynucleotide sequence of about 90% to 99% sequence identity to a Zip8 polynucleotide having the polynucleotide sequence of SEQ ID NO: 2, 3, or 5. The Zip8 polynucleotide can have at least about 91%, 92%, 93%, 94%, 95%, 96%, 97%, 98%, 99%, or 100% sequence identity to the polynucleotide sequence of SEQ ID NO: 2, 3, or 5.

### Promoters

The Zip14 constructs described herein can comprise a promoter operably coupled to a gene encoding Zip14, optionally Slc39a14. The promoter can be a ubiquitous promoter, optionally Rosa or CAG. The promoter can be a tissue specific promoter. For example, the Zip14 constructs described herein can comprise a tissue specific promoter operably coupled to a gene encoding Zip14, optionally Slc39a14. The tissue specific promoter can be a neuron-specific promoter, an astrocyte specific promoter, a microglia specific promoter, a blood vessel specific promoter, or cardiomyocytes. Neuron-specific promoters include but are not limited to hSyn, Synapsin, Calbindin, Pax6, Sox2, Arc, neurofilament heavy chain (NEFH), or cFos. Astrocyte-specific promoters include but are not limited to GFAP and S100b. The microglia-specific promoters include but are not limited to Iba1. Blood vessel specific promoters include but are not limited to CD31. Cardiomyocytes specific promoters include but are not limited to alpha-myosin heavy chain or beta-myosin heavy chain.

The Zip8 constructs described herein can comprise a promoter operably coupled to a gene encoding Zip8, optionally SLC39A8. The promoter can be a ubiquitous promoter, optionally Rosa or CAG. The promoter can be a tissue specific promoter. For example, the Zip8 constructs described herein can comprise a tissue specific promoter operably coupled to a gene encoding Zip8, optionally SLC39A8. The tissue specific promoter can be a neuron-specific promoter, an astrocyte specific promoter, a microglia specific promoter, a blood vessel specific promoter, or cardiomyocytes. Neuron-specific promoters include but are not limited to hSyn, Synapsin, Calbindin, Pax6, Sox2, Arc, neurofilament heavy chain (NEFH), or cFos. Astrocyte-specific promoters include but are not limited to GFAP and S100b. The microglia-specific promoters include but are not limited to Iba1. Blood vessel specific promoters include but are not limited to CD31. Cardiomyocytes specific promoters include but are not limited to alpha-myosin heavy chain or beta-myosin heavy chain.

### Vectors

The Zip14 constructs described herein can comprise a vector. The vector can be a viral or non-viral vector.

Non-viral vectors include but are not limited to plasmids, Bacterial Artificial Chromosomes (BAC), Yeast Artificial Chromosomes (YAC). The Zip14 constructs described herein can comprise a non-viral vector can be delivered using lipid nanoparticles. The Zip14 constructs described herein can comprise a non-viral vector and be delivered as naked DNA. The Zip14 constructs described herein can comprise a non-viral vector can also be formulated into a polymer carrier.

The viral vector can be an Adeno-associated viral vector (AAV), adenoviral vector (AdV), Lentiviral Vector (LVV). Serotypes of AAV include but are not limited to AAV1, AAV2, AAV3, AAV4, AAV5, AAV6, AAV7, AAV8, and AAV9. These serotypes differ in their tropism, or the types of cells they infect, making AAV a very useful system for preferentially transducing specific cell types. AAV1, AAV2, AAV4, AAV5, AAV8, and AAV9 are the preferred serotypes for targeting Central Nervous System (CNS) tissue. AAV1, AAV8, and AAV9 are the preferred serotypes for targeting cardiac tissue. AAV2 is the preferred serotype for targeting kidneys. AAV7, AAV8, and AAV9 is the preferred serotypes for targeting the liver. AAV4, AAV5, AAV6, and AAV9 is the preferred serotypes for targeting the lungs. AAV8 is the preferred serotypes for targeting the pancreas. AAV2, AAV5, and AAV8 is the preferred serotypes for targeting photoreceptor cells. AAV1, AAV2, AAV4, AAV5, and AAV8 is the preferred serotypes for targeting RPE (Retinal Pigment Epithelium). AAV1, AAV6, AAV7, AAV8, and AAV9 is the preferred serotypes for targeting skeletal muscle tissue. AAV9 and retroAAV9 are preferred viral vectors for the Zip14 constructs described herein.

Other serotypes of adeno-associated viruses (AAV) include but are not limited to PHP.eB, PHP.S, and PHP.V1. The PHP.eB serotype exhibits transduction of central nervous system tissue via systemic delivery. The PHP.S serotype exhibits transduction of peripheral nervous system tissue via systemic delivery. The PHP.V1 serotype exhibits transduction of vesicular brain cells via systemic delivery.

Depending on the delivery route and/or intention for the viral vector to move anterograde or retrograde, different viral vectors can be used. For example, AAV9 is an anterograde virus. retroAAV is a retrograde virus. PHPeB is an AAV serotype that can be delivered intravenously and deliver the construct to the brain.

### Compositions

The Zip14 constructs described herein can be formulated into a composition. The compositions comprising a Zip14 constructs described herein can be a pharmaceutical composition. The composition, including pharmaceutical compositions, may comprise an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

The compositions described herein can comprise a single Zip14 construct described herein or a combination of at least two Zip14 constructs. The compositions described herein can comprise a mixture of different Zip14 constructs.

The compositions described herein can be formulated as a pharmaceutical composition comprising a Zip14 constructs described herein and pharmaceutically acceptable carrier. Pharmaceutically acceptable carriers include, but are not limited to, excipient, lubricant, emulsifier, stabilizer, solvent, diluent, buffer, vehicle, or a combination thereof.

Pharmaceutically acceptable carriers are well known in the art and include, for example, aqueous solutions such as water or physiologically buffered saline or other solvents or vehicles such as glycols, glycerol, oils such as olive oil or injectable organic esters. Pharmaceutically acceptable carriers can be a liquid, including but not limited to water and oils, including those of petroleum, animal, vegetable or synthetic origin, such as peanut oil, soybean oil, mineral oil, and sesame oil. The pharmaceutical carriers can be saline, gum acacia, gelatin, starch paste, talc, keratin, colloidal silica, or urea. In addition, auxiliary, stabilizing, thickening, lubricating and coloring agents can be used. For example, sugars and/or amino acids can be admixed into the pharmaceutical composition. The pharmaceutical composition may comprise water, glycerin, phospholipids, or a mixture thereof. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002).

The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

The pharmaceutical compositions can include the formulation of the Zip14 constructs described herein with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component, e.g., a Zip14 constructs described herein, or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Single or multiple administrations of the compositions can be carried out with dose levels and pattern being selected by the treating physician. In any event, the pharmaceutical formulations should provide a quantity of a Zip14 constructs described herein sufficient to effectively inhibit treat an indication, either therapeutically or prophylactically.

### Delivery—Routes of Administration

The Zip14 constructs described herein can be administered subcutaneously, intramuscularly, intravenously, intraperitoneally, intrapleurally, intravesicularly, intrathecally, or by a route as necessitated condition. The compositions comprising a Zip14 constructs described herein can be infused into a subject. The composition comprising Zip14 constructs described herein can be administered by parenteral administration. One route of administration is intravenous. A route of administration is intracerebroventricular injection. Intranasal injection may also be used.

### Nucleic Acids, Vectors, and Host Cells

The present invention also provides for an isolated nucleic acid molecule encoding a Zip14 constructs described herein. Nucleic acid molecules that encode the Zip14 constructs described herein. The nucleic acids can be present in whole cells, in a cell lysate, or in a partially purified or substantially pure form. A nucleic acid can be isolated by purification away from other cellular components or other contaminants (e.g. other cellular nucleic acids or proteins) by standard techniques, including alkaline/SDS treatment, CsCl banding, column chromatography, agarose gel electrophoresis and others well known in the art. See Ausubel, et al. (2011) Current Protocols in Molecular Biology John Wiley & Sons, Inc. A nucleic acid described herein can be, for example, DNA or RNA and may or may not contain intronic sequences. The nucleic acid can be a cDNA molecule. Nucleic acids described herein can be obtained using standard molecular biology techniques. Specifically, degenerate codon substitutions can be achieved by generating, e.g., sequences in which the third position of one or more selected codons is substituted with mixed-base and/or deoxyinosine residues. Batzer, et al. (1991) Nucleic Acid Res. 19:5081; Ohtsuka, et al. (1985) J. Biol. Chem. 260:2605-08; Rossolini, et al. (1994) Mol. Cell. Probes 8:91-98.

### Tissue Visualization

The Zip14 constructs described herein can be used for tissue visualization, tissue expression, tissue mapping, monitoring trauma, and/or tracking gene therapy delivery and expression.

The inventors surprisingly discovered that the Zip14 constructs described herein can be used for in applications for visualization of tissue. Without wishing to be bound to a theory, the Zip14 constructs described herein overexpress Zip14 protein. The Zip14 protein concentrates Manganese (Mn²⁺) allowing for non-invasive visualization via MRI. The Zip14 constructs described herein can be administered to a subject and used to count cells, determine cell density, detect anatomical anomalies, monitor trauma, assess trauma (injury), monitor the progress of a degenerative disease.

The Zip14 constructs described herein can be administered to a patient and the Zip14 constructs described herein used for tissue visualization, tissue expression, tissue mapping, monitoring trauma, and/or tracking gene therapy delivery and expression via imaging methods, e.g., Magnetic Resonance Imaging (MRI). The imaging methods include but are not limited to Magnetic Resonance Imaging (MRI), computerized tomography (CT) scans, Positron Emission Tomography (PET).

For example, the Zip14 constructs described herein can be administered to a subject and the subject examined by imaging methods. The Zip14 constructs described herein can be visualized by imaging methods to allow for tissue visualization, tissue expression, tissue mapping, monitoring trauma, and/or tracking gene therapy delivery and expression.

The Zip14 constructs described herein can be administered systematically via intravenous injection. The Zip14 constructs described herein can cross the blood-brain barrier, and thus Zip14 constructs described herein administered systematically can reach the central nervous system, e.g., brain.

Additionally, the Zip14 constructs described herein can administered to the brain, optionally the cerebellum or the cerebrum, heart, lung, liver, stomach, small intestine, optionally the duodenum, the jejunum, the ileum, large intestine, optionally the ascending colon, the transverse colon, the descending colon, pancreas, kidney, the blood stream, a bone, or a muscle.

The methods described herein can further comprise using machine learning to map the area detectable by Zip14 expression. The machine learning can be trained on controls and existing data of Zip14 expression tests. The machine learning classifies the data to produce a method of distinguishing areas with Zip14 expression from those without Zip14 expression. This data is used to map the tissue, identify trauma, evaluate disease progression, or a combination thereof. Machine learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, deep neural networks, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof, can be used to classify the data. See e.g., Han & Kamber (2006) Chapter 6, Data Mining, Concepts and Techniques, 2nd Ed. Elsevier: Amsterdam. As described herein, any classifier or combination of classifiers (e.g., ensemble) may be used in a classification system. The data collected from the imaging step can be used to train a classifier.

### Vectors for Gene Delivery

For delivery to a cell or organism, a polynucleotide encoding an Zip14 constructs described herein can be incorporated into a vector. Examples of vectors used for such purposes include expression plasmids capable of directing the expression of the nucleic acids in the target cell. The vector can be a viral vector system wherein the polynucleotide is incorporated into a viral genome that is capable of transfecting the target cell. In one embodiment, the encoding polynucleotide can be operably linked to expression and control sequences that can direct expression of the polypeptide or oligonucleotide in the desired target host cells. Thus, one can achieve expression of the polypeptide or oligonucleotide inhibitor under appropriate conditions in the target cell.

### Gene Delivery Systems

Viral vector systems useful in the expression of a Zip14 constructs described herein include, but are not limited to, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, optionally AAV-PHP.eB, minute virus of mice (MVM), HIV, sindbis virus, and retroviruses (including but not limited to Rous sarcoma virus and lentivirus), and MoMLV.

Adeno-associated viral (AAV) vectors are preferred for central nervous system (CNS) gene therapy because these vectors can cross the blood-brain barrier (BBB). For example, the AAV-PHP.eB vector can be used in methods requiring crossing the blood-brain barrier (BBB) to reach CNS cells for therapy. The inventor surprisingly found that the Zip14 constructs described herein can be administered intravenously to the circulatory system and cross the blood-brain barrier.

Typically, the coding sequence of interest (e.g., one encoding for a Zip14 constructs described herein) are inserted into such vectors to allow packaging of the gene construct, typically with accompanying viral DNA, followed by infection of a sensitive host cell and expression of the coding sequence of interest.

A gene delivery system can be any means for the delivery of a polynucleotide sequence encoding a Zip14 constructs described herein to a target cell. The nucleic acids can be conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180), or by ultrasound-microbubble delivery system (Lan H Y et al., J. Am Soc. Nephrol. 14:1535-1548). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells. See, e.g., WO 93/20221, WO 93/14188, and WO 94/06923. The DNA constructs encoding a Zip14 constructs described herein can be linked to viral proteins, such as adenovirus particles, to facilitate endocytosis (Curiel et al., Proc. Natl. Acad. Sci. U.S.A. 88:8850-8854 (1991)), microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a Zip14 constructs described herein into target cells or patients. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site).

The design of retroviral vectors is known in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in the art. European Patent Application 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide inhibitor of the invention and thus restore the target cells (e.g., erythroid cells) to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins can be used. Alternatively, amphotropic or xenotropic envelope proteins, such as those produced by PA317 and GPX packaging cell lines can be used to package the retroviral vectors.

### Administration of Compositions

Compositions comprising Zip14 constructs described herein or a polynucleotide sequence encoding a Zip14 constructs described herein can be delivered to target tissue or organ using any delivery method known in the art. For example, the Zip14 constructs described herein can be administration via a subcutaneous, intramuscular, intravenous, intraperitoneal, intrapleural, intravesicular, intrathecal, intracerebroventricular, intranasal injection, or a combination thereof.

The compositions described herein can be administered to a cell. The cell can be provided as part of a tissue, such as erythrocytes as a part of the circulatory system, or as an isolated cell, such as in tissue culture. The cell can be provided in vivo, ex vivo, or in vitro.

The compositions described herein can be introduced into the tissue of interest in vivo or ex vivo by a variety of methods. Nucleic acids encoding a Zip14 constructs described herein, or a Zip14 constructs described herein, can be introduced into cells by such methods as microinjection, calcium phosphate precipitation, liposome fusion, ultrasound, electroporation, biolistics, or a combination thereof.

The Zip14 constructs described herein can be administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector.

Doses may generally range between about working titers of 1×10¹⁰ vg/mL (viral genome per mL) and 1×10¹⁴ vg/mL Zip14 constructs described herein. The dosage can be about 1×10¹⁰ vg/mL, 1×10¹¹ vg/mL, 1×10¹² vg/mL, 1×10¹³ vg/mL, or 1×10¹⁴ vg/mL ZIP14 constructs described herein. The dosage can be about 1×10¹² vg/mL Zip14 constructs described herein.

### Kits

The invention also provides kits for Zip14 constructs are also described. The kits typically include a container that contains (1) a pharmaceutical composition having an effective amount of an Zip14 constructs described herein and (2) informational material containing instructions on how to dispense the pharmaceutical composition, including description of the type of patients who can be treated (e.g., human patients suffering from loss of function mutations in Slc39a14—the gene coding for the protein Zip14), the schedule (e.g., dose and frequency) and route of administration. In some cases, a second container is included in the kit to provide a second pharmaceutical composition comprising an effective amount of a second active agent.

### EXAMPLES

### Example 1

### MRI Detection of Gene Expression in the Mammalian Brain Using the Manganese Transporter Zip14

### Synopsis

The inventors used Zip14, a Manganese transporter, as an MRI-visible gene expression reporter system. Mice were intracranially injected into various brain areas with AAVs carrying a plasmid construct of Synapsin promoter for neuronal-specific expression, Zip14, and histological marker. MRI was performed immediately after, 2 weeks, and 4 weeks after injection. Significant, large magnitude hyperintensity was observed 2 and 4 weeks after injection, but not immediately after, at the injection site and areas projecting away or towards the injection site. This gene expression reporter system is viable without the use of additional contrast agents or non-standard imaging protocols.

### Introduction

There has been considerable interest in developing genetically encoded reporters that alter MRI contrast for imaging in the mammalian brain. Many groups have exploited a variety of proteins to alter contrast mechanisms. Despite significant efforts, these techniques have been not widely adopted. This could be due to difficulty in contrast agent delivery to the brain and/or high overexpression requirements of exogenous proteins. Without wishing to be bound to a theory, the inventors used that the metal transporter gene Slc39a14 coding for the protein Zip14 has potential as an MRI reporter system. Zip14 is highly expressed in the liver where it regulates blood Mn levels. Patients with loss-of-function mutations in Slc39a14 exhibit hyperintensity on T1w MRI of the brain due to increased blood Manganese (Mn) concentration. Additionally, hyperintensity in Mn-enhanced MRI of the brain correlates with areas expressing higher levels of Zip14. The inventors demonstrated that Zip14 overexpression in the brain using viral delivery produces long lasting, specific signal enhancement in vivo.

### Methods

The plasmid construct used in this study consisted of a Synapsin promoter for neuronal specific expression, Slc39a14 isoform 1, and a histology visible tag (either EGFP or FLAG) designed to be added to the C-terminus of ZIP14. The plasmid was packaged into either AAV9 or retroAAV capsids for anterograde (AAV-Zip14) or retrograde (retroAAV-Zip14) labeling, respectively, with final working titers of 1×10¹² vg/mL (viral genome per mL).

Separate stereotaxic injections were performed on adult C57B16J mice at coordinates corresponding to the geometric centers of the S1 barrel cortex (S1BC), ventro posteromedial nucleus (VPM), or the caudate putamen (CP).

MRI was performed on a 11.7 T animal MRI system (Magnex Scientific Magnet/Bruker Electronics) using a CryoProbe system (Bruker). Scan protocol included a 1 min low-resolution pilot and an 80 μm isotropic resolution MDEFT sequence [TE=3.6 ms, TR=4 s, TI=1100 ms]. Imaging was performed at 0-, 2-, and 4-weeks post injection (±2 days).

After the last imaging session, animals were transcardially perfused with paraformaldehyde and cryoprotected in 30% sucrose for histology. MRI were skull stripped using 3DPCNN. Allen Mouse Brain Atlas (CCFv3) labels were registered to the image native space using ANTs. Further image analyses were performed using Python, MIPAV, and 3D Slicer. Statistical analyses were performed using R and GraphPad Prism.

### Results

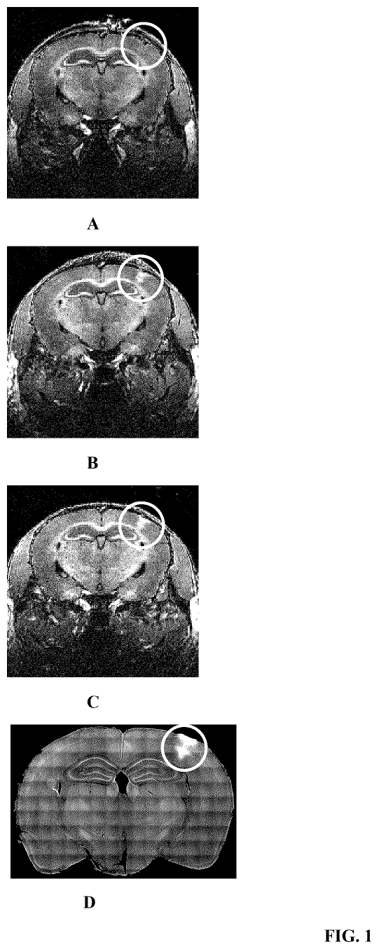

At 0 weeks post injection of AAV-Zip14-EGFP, no visually apparent differences in signal were observed in the S1BC (FIG. 1A). However, at 2 weeks post injection, there was marked hyperintensity in the S1BC with heterogeneity in intensity across the cortical layers (FIG. 1B). This signal enhancement pattern persisted through 4 weeks post injection (FIG. 1C). Immunohistochemistry validated that the MRI signal enhancement observed in the S1BC was due to Zip14 overexpression (FIG. 1D). Well established neurotropism of AAV9 could explain the lack of MRI contrast and signal in immunohistology in layer 4. Comparison with matched histology revealed expression of Zip14 in the cell bodies and dendrites in the S1BC.

MRI signal enhancement in the S1BC at each timepoint was quantified. After thresholding to the top 10% intensity voxels, no significant differences in signal were observed at 0 weeks post injection. Significant signal increases were observed the injection side S1BC compared to control at 2 and 4 weeks post injection.

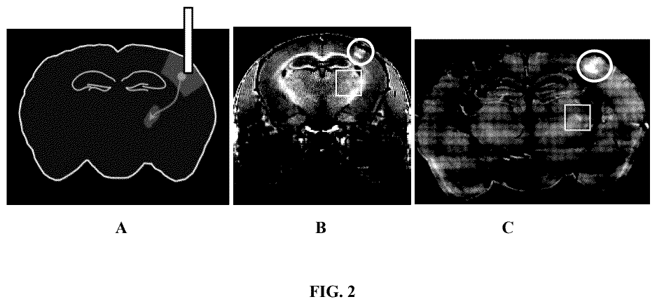

MRI enhancement could be detected in the processes of neurons that project from the S1BC to the thalamus (FIG. 2A, 2B). Histology confirmed Zip14-EGFP-positive processes in the thalamus, but not cell bodies (FIG. 2C). This indicates that the Zip14 moved anterograde down axons to the thalamus and caused MRI enhancement in the thalamus. Injections of AAV-Zip14-EGFP into the VPM of the thalamus produced focal MRI signal enhancement in layer 4 of somatosensory areas of the cortex. Histology showed Zip14 overexpression in cell bodies and dendrites in the thalamus, and focal overexpression in the processes in cortical layer 4 as expected from anterograde transport from the injection site.

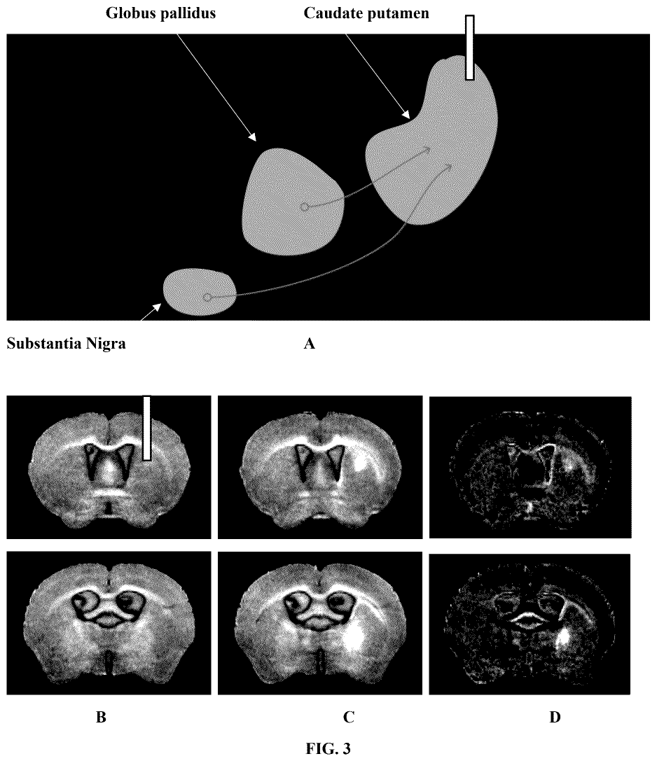

To determine if MRI enhancement could occur with retrograde viruses, retroAAV-Zip14-FLAG was injected into the CP (FIG. 3A). No significant MR signal enhancement at the injection site or posterior brain areas at 0 weeks post injection was detected (FIG. 3B). At 2 weeks post injection, focal hyperintensity was apparent at the injection site and several posterior areas (FIG. 3C). Subtracting the 2 week post injection from the 0 week post injection image and rendering in 3D allowed the sites of enhancement to be visualized at the injection site (CP) and areas projecting to the CP such as globus pallidus, pallidus, and substantia nigra (FIG. 3D).

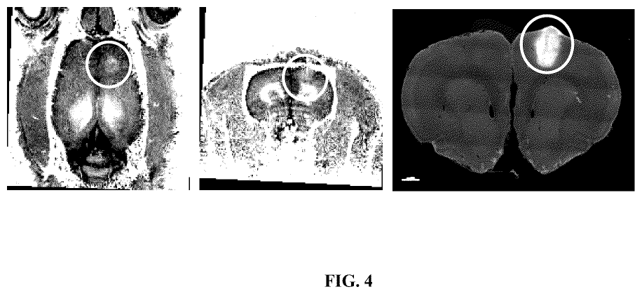

To demonstrate the usefulness in regenerative medicine, transplanted cortical precursor cells were injected into a region of rodent brain cortex that had been ablated similar to a traumatic brain injury. AAV-Zip14-EGFP virus was injected into the tissue grown from these cells, enhancement of this newly formed tissue could be detected (FIG. 4A, 4B). Histological staining for the precursor cells and Zip14-EGFP confirmed that the enhancement was due to overexpression of Zip14 in the implanted precursor cells (FIG. 4C). Furthermore, when anterograde and retrograde Zip14 viruses were injected projection being formed by the new tissue and the existing brain could be detected.

### CONCLUSION

Zip14 overexpression is an MRI-visible expression reporter system. The signal enhancement observed in our experiments was obtained without the use of additional contrast agents and without sophisticated image acquisition beyond vendor supplied sequences. The neural tracing application of AAV-Zip14 and retroAAV-Zip14 could be readily extended to other brain areas or used in the context of neurodevelopment, brain injury, or neurodegeneration models. Even at high levels of expression, modification of this construct to include other cell type-specific promoters or co-expression with other genes of interest allows for the use of Zip14 in reporter applications to other cell types and organ systems.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. An isolated polynucleotide comprising a tissue-specific promoter operably coupled to a Zip14 encoding sequence or a Zip8 encoding sequence.

2. The polynucleotide of claim 1, wherein the Zip14 sequence comprises a polynucleotide with at least about 90% sequence identity to the polynucleotide sequence of SEQ ID NO:1.

3-4. (canceled)

5. The polynucleotide of claim 1, wherein the Zip14 sequence, or Zip8 encoding sequence, is codon-optimized, optionally for humans.

6. (canceled)

7. The polynucleotide of claim 1, wherein the Zip8 sequence comprise a polynucleotide with at least about 90% homology to the polynucleotide sequence of SEQ ID NO: 2, 3, or 5.

8-11. (canceled)

12. The polynucleotide of claim 1, wherein the polynucleotide further comprises a reporter gene, optionally a fluorescent protein.

13. (canceled)

14. The polynucleotide of claim 1, wherein the tissue-specific promoter is a neuronal specific promoter, optionally a Synapsin, Calbindin, Pax6, Sox2, Arc, neurofilament heavy chain (NEFH), or cFos promoter, an astrocyte specific promoter, optionally a GFAP or S100b promoter, a microglia specific promoter, optionally Iba1 promoter, a blood vessel specific promoter, optionally a Rosa or CAG promoter, cardiomyocyte specific promoter, optionally an alpha-myosin heavy chain promoter or beta-myosin heavy chain promoter, or a skeletal muscle specific promoter, optionally a myosin heavy chain 1 (MYH1) promoter or actinin alpha 3 (ACTN3) promoter.

15-26. (canceled)

27. An isolated vector comprising the polynucleotide of claim 1, optionally wherein the vector is a Bacterial Artificial Chromosome (BAC), Yeast Artificial Chromosome (YAC), or a plasmid or wherein the vector is a viral vector, optionally a retroviral vector, an adenoviral vector (AdV), an adeno-associated viral vector (AAV), or a lentiviral vector.

28-29. (canceled)

30. The vector of claim 27, wherein the vector is formulated with lipid nanoparticles.

31. The vector of claim 27, wherein the vector is a viral vector.

32. The vector of claim 31, wherein the vector is a retroviral vector, an adenoviral vector (AdV), an adeno-associated viral vector (AAV), or a lentiviral vector.

33. The vector of claim 32, wherein the vector is a AAV1, AAV2, AAV3, AAV4, AAV5, AAV6, AAV7, AAV8, a AAV9 serotype adenoviral-associated vector, PHP.eB, PHP.S, or PHP.V1 serotype AAV vector.

34-37. (canceled)

38. A method of monitoring gene expression in a subject comprising administering a composition comprising an effective amount the polynucleotide of claim 1 to a subject in need thereof and evaluating the subject using an imaging method.

39. (canceled)

40. The method of claim 38, wherein the method further comprises determining cell number, cell density, cell health, cell death, or a combination thereof.

41. The method of claim 38, wherein the method further comprises tissue visualization, tissue expression, tissue mapping, monitoring trauma, and/or tracking gene therapy delivery and expression via the imaging method.

42. The method of claim 38, wherein the imaging method is selected from Magnetic Resonance Imaging (MRI), computerized tomography (CT) scans, Positron Emission Tomography (PET), or a combination thereof.

43. (canceled)

44. The method of claim 38, wherein the administration is subcutaneous, intramuscular, intravenous, intraperitoneal, intrapleural, intravesicular, intrathecal, intracerebroventricular, intranasal injection, or a combination thereof.

45. The method of claim 44, wherein the administration is intravenous.

46. (canceled)

47. The method of claim 38, wherein the composition comprises between about 1×10¹⁰ vg/mL (viral genome per mL) and 1×10¹⁴ vg/mL of the vector.

48-58. (canceled)

59. The method of claim 38, wherein the method further comprises using machine learning to map the area detectable by ZIP14 expression.

60. The method of claim 59, wherein the machine learning classifier is AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, deep neural networks, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof.


SEQUENCE LISTING (4 sequences)

SEQ ID NO: 1 - DNA, 1476 bp, Mus musculus
  source 1..1476: mol_type = other DNA
atgaagctgctgctgctgcacccggccttccagagctgcctcctgctgaccctgcttggcttatggagaaccacccctgaggctcacgcttcatccctgggtgcaccagctatcagcgctgcctccttcctgcaggatctaatacatcggtatggcgagggtgacagcctcactctgcagcagctgaaggccctactcaaccacctggatgtgggagtgggccggggtaatgtcacccagcacgtgcaaggacacaggaacctctccacgtgctttagttctggagacctcttcactgcccacaatttcagcgagcagtcgcggattgggagcagcgagctccaggagttctgccccaccatcctccagcagctggattcccgggcctgcacctcggagaaccaggaaaacgaggagaatgagcagacggaggaggggcggccaagcgctgttgaagtgtggggatacggtctcctctgtgtgaccgtcatctccctctgctccctcctgggggccagcgtggtgcccttcatgaagaagaccttttacaagaggctgctgctctacttcatagctctggcgattggaaccctctactccaacgccctcttccagctcatcccggaggcatttggtttcaaccctctggaagattattatgtctccaagtctgcagtggtgtttgggggcttttatcttttctttttcacagagaagatcttgaagattcttcttaagcagaaaaatgagcatcatcatggacacagccattatgcctctgagtcgcttccctccaagaaggaccaggaggagggggtgatggagaagctgcagaacggggacctggaccacatgattcctcagcactgcagcagtgagctggacggcaaggcgcccatggtggacgagaaggtcattgtgggctcgctctctgtgcaggacctgcaggcttcccagagtgcttgctactggctgaaaggtgtccgctactctgatatcggcactctggcctggatgatcactctgagcgacggcctccataatttcatcgatggcctggccatcggtgcttccttcactgtgtcagttttccaaggcatcagcacctcggtggccatcctctgtgaggagttcccacatgagctaggagactttgtcatcctgctcaacgctgggatgagcatccaacaagctctcttcttcaacttcctttctgcctgctgctgctacctgggtctggcctttggcatcctggccggcagccacttctctgccaactggatttttgcgctagctggaggaatgttcttgtatatttctctggctgatatgttccctgagatgaatgaggtctgtcaagaggatgaaaggaagggcagcatcttgattccatttatcatccagaacctgggcctcctgactggattcaccatcatggtggtcctcaccatgtattcaggacagatccagattggg

SEQ ID NO: 2 - DNA, 3930 bp, Homo sapiens
  source 1..3930: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagcaggctccaccatggcccctggccgggccgtggccggcctgctgctgctggcagcagcaggactgggaggagtggccgagggcccaggcctggccttttccgaggacgtgctgagcgtgttcggagcaaacctgtccctgtctgccgcacagctgcagcacctgctggagcagatgggagcagcatccagagtgggagtgccagagcctggccagctgcacttcaaccagtgtctgaccgccgaggagatctttagcctgcacggcttctccaatgccacccagatcacaagctccaagttttctgtgatctgccccgccgtgctgcagcagctgaacttccacccgtgcgaggataggccgaaacataagacaagacctagccactccgaagtgtggggctacggctttctgtctgtgaccatcatcaatctggccagcctgctgggcctgatcctgacaccactgatcaagaagtcctatttccccaagatcctgaccttctttgtgggcctggccatcggcacactgttttctaacgccatcttccagctgatccccgaggcctttggcttcgaccctaaggtggatagctacgtggagaaggccgtggccgtgtttggcggcttctatctgctgttctttttcgagaggatgctgaagatgctgctgaagacctacggccagaatggccacacacactttggcaacgacaatttcggcccccaggagaagacccaccagccaaaggccctgccagccatcaacggagtgacctgctatgccaatcctgccgtgacagaggccaacggccacatccactttgacaacgtgagcgtggtgtctctgcaggatggcaagaaggagccttctagctgcacctgtctgaagggcccaaagctgtccgagatcggcaccatcgcctggatgatcacactgtgcgacgccctgcacaacttcatcgatggcctggccatcggcgcctcttgtaccctgagcctgctgcagggcctgtctacaagcatcgccatcctgtgcgaggagtttccccacgagctgggcgacttcgtgatcctgctgaacgccggcatgtccaccagacaggccctgctgtttaatttcctgagcgcctgctcctgttacgtgggactggccttcggcatcctggtgggcaacaattttgccccaaatatcatcttcgccctggccggcggcatgtttctgtatatcagcctggccgacatgttccccgagatgaacgatatgctgagggagaaggtgaccggccgcaagaccgacttcaccttcttcatgatccagaatgccggcatgctgaccggcttcacagccatcctgctgatcacactgtacgccggcgagatcgagctggaggacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 3 - DNA, 1380 bp, synthetic construct
  source 1..1380: mol_type = other DNA
atggcccctggccgggccgtggccggcctgctgctgctggcagcagcaggactgggaggagtggccgagggcccaggcctggccttttccgaggacgtgctgagcgtgttcggagcaaacctgtccctgtctgccgcacagctgcagcacctgctggagcagatgggagcagcatccagagtgggagtgccagagcctggccagctgcacttcaaccagtgtctgaccgccgaggagatctttagcctgcacggcttctccaatgccacccagatcacaagctccaagttttctgtgatctgccccgccgtgctgcagcagctgaacttccacccgtgcgaggataggccgaaacataagacaagacctagccactccgaagtgtggggctacggctttctgtctgtgaccatcatcaatctggccagcctgctgggcctgatcctgacaccactgatcaagaagtcctatttccccaagatcctgaccttctttgtgggcctggccatcggcacactgttttctaacgccatcttccagctgatccccgaggcctttggcttcgaccctaaggtggatagctacgtggagaaggccgtggccgtgtttggcggcttctatctgctgttctttttcgagaggatgctgaagatgctgctgaagacctacggccagaatggccacacacactttggcaacgacaatttcggcccccaggagaagacccaccagccaaaggccctgccagccatcaacggagtgacctgctatgccaatcctgccgtgacagaggccaacggccacatccactttgacaacgtgagcgtggtgtctctgcaggatggcaagaaggagccttctagctgcacctgtctgaagggcccaaagctgtccgagatcggcaccatcgcctggatgatcacactgtgcgacgccctgcacaacttcatcgatggcctggccatcggcgcctcttgtaccctgagcctgctgcagggcctgtctacaagcatcgccatcctgtgcgaggagtttccccacgagctgggcgacttcgtgatcctgctgaacgccggcatgtccaccagacaggccctgctgtttaatttcctgagcgcctgctcctgttacgtgggactggccttcggcatcctggtgggcaacaattttgccccaaatatcatcttcgccctggccggcggcatgtttctgtatatcagcctggccgacatgttccccgagatgaacgatatgctgagggagaaggtgaccggccgcaagaccgacttcaccttcttcatgatccagaatgccggcatgctgaccggcttcacagccatcctgctgatcacactgtacgccggcgagatcgagctggag

SEQ ID NO: 4 - protein, 460 aa, synthetic construct
MAPGRAVAGLLLLAAAGLGGVAEGPGLAFSEDVLSVFGANLSLSAAQLQHLLEQMGAASRVGVPEPGQLHFNQCLTAEEIFSLHGFSNATQITSSKFSVICPAVLQQLNFHPCEDRPKHKTRPSHSEVWGYGFLSVTIINLASLLGLILTPLIKKSYFPKILTFFVGLAIGTLFSNAIFQLIPEAFGFDPKVDSYVEKAVAVFGGFYLLFFFERMLKMLLKTYGQNGHTHFGNDNFGPQEKTHQPKALPAINGVTCYANPAVTEANGHIHFDNVSVVSLQDGKKEPSSCTCLKGPKLSEIGTIAWMITLCDALHNFIDGLAIGASCTLSLLQGLSTSIAILCEEFPHELGDFVILLNAGMSTRQALLFNFLSACSCYVGLAFGILVGNNFAPNIIFALAGGMFLYISLADMFPEMNDMLREKVTGRKTDFTFFMIQNAGMLTGFTAILLITLYAGEIELE